(19) United States
(12) Patent Application Publication
(10) Pub. No.: US 2023/0192637 A1
(43) Pub. Date: Jun. 22, 2023

(54) HYDROGENATION OF NEPETALACTONE

(71) Applicant: Zymergen Inc., Emeryville, CA (US)

(72) Inventors: Szymon Kosinski, San Francisco, CA (US); Liza Lopez, San Jose, CA (US); Wilson Chau, Oakland, CA (US); Anil Guram, Thousand Oaks, CA (US); Ying Lin Louie, Oakland, CA (US)

(73) Assignee: Zymergen Inc., Emeryville, CA (US)

(21) Appl. No.: 17/995,920

(22) PCT Filed: Apr. 14, 2021
(86) PCT No.: PCT/US2021/027362

§ 371 Date: Oct. 10, 2022

Related U.S. Application Data
(60) Provisional application No. 63/010,466, filed on Apr. 15, 2020.

(51) Int. Cl. C07D 309/10; B01J 23/755

(52) U.S. Cl. CPC C07D 309/10; B01J 23/755

(57) ABSTRACT

A process for the production of dihydronepetalactone including hydrogenating nepetalactone in the presence of a catalytic metal is provided. The catalytic metal may include nickel. The process may be performed in an aqueous medium.

### CROSS-REFERENCE TO RELATED APPLICATION

This application claims the benefit of U.S. Provisional Application No. 63/010,466, filed Apr. 15, 2020, which is incorporated by reference in its entirety.

### FIELD OF THE DISCLOSURE

The present disclosure relates generally to processing nepetalactone. More specifically, this disclosure relates to hydrogenation of nepetalactone to dihydronepetalactone.

### BACKGROUND

Nepetalactone is an effective active ingredient for insect repellents, which may be produced using engineered microbial cells. Dihydronepetalactone is a more potent insect repellent than nepetalactone and may be produced by hydrogenation of nepetalactone.

### SUMMARY

In a first aspect, a process for the production of dihydronepetalactone is provided. The process includes hydrogenating of formula (II) comprising hydrogenating a nepetalactone of formula (I) in presence of a catalytic metal that includes nickel according to the following scheme:

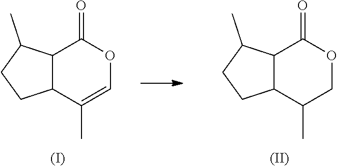

At least 20%, at least 30%, at least 40%, at least 50%, at least 60%, at least 70%, at least 80%, at least 85%, at least 90%, at least 92%, at least 94%, at least 95%, at least 96%, at least 97%, at least 98%, at least 98.5%, at least 99%, at least 99.5%, at least 99.7%, at least 99.8%, at least 99.9%, at least 99.95%, at least 99.99%, or at least 100% of the nepetalactone is converted. In some embodiments, the hydrogenation is substantially complete, with no detectable nepetalactone present after hydrogenation.

The catalytic metal can be selected from an unsupported catalytic metal, a nickel alloy, elemental nickel, or any combination thereof. The catalytic metal can consist essentially of a nickel-aluminum alloy.

The dihydronepetalactone can be formed with a selectivity of at least 50%, at least 60%, at least 70%, at least 75%, at least 80%, at least 85%, at least 90%, at least 91%, at least 92%, at least 93%, at least 94%, or at least 95%.

The hydrogenating can be performed in the presence of a solvent.

The solvent can be water, an alcohol, an amide, an alkane, an ester, or an ether. Examples include ethyl acetate, butyl acetate, di methylacetamide, ethanol, isopropyl alcohol, 1-butyl alcohol, 2-butyl alcohol, and dimethoxyethane. The hydrogenating can be performed without a solvent.

The hydrogenating can be effected in the presence of a metal promoter. The metal promoter can be tin, copper, gold, silver, molybdenum, iron, and combinations thereof.

The hydrogenating can be performed at a temperature of about 25° C. to about 250° C. or a temperature of about 50° C. to about 150° C. The hydrogenating can be performed at a pressure of about 0.1 MPa to about 20 MPa.

The nepetalactone of Formula (I) can be at least 85% cis/trans nepetalactone.

In a second aspect, a process for the production of dihydronepetalactone is provided. The process includes hydrogenating of formula (II) comprising hydrogenating in an aqueous medium a nepetalactone of formula (I) in presence of a catalytic metal according to the following scheme:

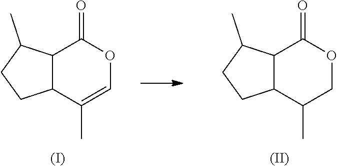

At least 20%, at least 30%, at least 40%, at least 50%, at least 60%, at least 70%, at least 80%, at least 85%, at least 90%, at least 92%, at least 94%, at least 95%, at least 96%, at least 97%, at least 98%, at least 98.5%, at least 99%, at least 99.5%, at least 99.7%, at least 99.8%, at least 99.9%, at least 99.95%, or at least 99.99% of the nepetalactone is converted.

The catalytic metal can be selected from an unsupported catalytic metal, a nickel alloy, elemental nickel, or any combination thereof. The catalytic metal can consist essentially of a nickel-aluminum alloy.

The dihydronepetalactone can be formed with a selectivity of at least 50%, at least 60%, at least 70%, at least 75%, at least 80%, at least 85%, at least 90%, at least 91%, at least 92%, at least 93%, at least 94%, or at least 95%.

The hydrogenating can be effected in the presence of a metal promoter. The metal promoter can be tin, copper, gold, silver, molybdenum, iron, and combinations thereof.

The hydrogenating can be performed at a temperature of about 25° C. to about 250° C. or a temperature of about 50° C. to about 150° C. The hydrogenating can be performed at a pressure of about 0.1 MPa to about 20 MPa.

The nepetalactone of Formula (I) can be at least 85% cis/trans nepetalactone.

### DETAILED DESCRIPTION

This written description uses examples to disclose the embodiments, including the best mode, and also to enable those of ordinary skill in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities may be performed in addition to those described. The order in which activities are listed is not necessarily the order in which they are performed.

In this specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Benefits, other advantages, and solutions to problems have been described herein with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment.

Conversely, various features that are, for brevity, described in the context of a single embodiment, may also be provided separately or in any subcombination. Further, references to values stated in ranges include each and every value within that range.

As a stated in the Summary, a process includes hydrogenating of formula (II) comprising hydrogenating a nepetalactone of formula (I) in presence of a catalytic metal according to the following scheme:

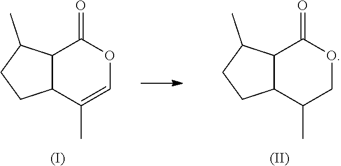

Nepetalactone (4,7-Dimethyl-5,6,7,7a-tetrahydrocyclopenta[c]pyran-1(4aH)-one (CAS No. 490-10-8)) is an effective active ingredient for insect repellent and can be produced, for example, by fermentation process using engineered cells. Production of nepetalactone is described in PCT Publication No. 2019126778, entitled “Nepetalactol Oxidoreductases, Nepetalactol Synthases, And Microbes Capable of Producing Nepetalactone,” which is incorporated by reference in its entirety and specifically for its description of producing nepetalactone. Nepetalactone may also be obtained from the plant catnip.

The term “nepetalactone” refers to any nepetalactone stereoisomer or mixture of nepetalactone stereoisomers. In some embodiments, nepetalactone may be provided as a single stereoisomer or as a mixture of stereoisomers. In some embodiments, any one of cis,trans-nepetalactone, trans,cis-nepetalactone, trans,trans-nepetalactone, and/or cis,cis-nepetalactone, or a mixture of any two or more thereof is provided.

Dihydronepetalactone (4,7-dimethyl-4,4a,5,6,7,7a-hexahydro-3H-cyclopenta[c]pyran-1-one) is an even more potent insect repellent than nepetalactone. The term “dihydronepetalactone” refers to any hydrogenated nepetalactone stereoisomer or mixture of dihydronepetalactone stereoisomers. In some embodiments, any one of cis,trans-dihydronepetalactone, trans,cis-dihydronepetalactone, trans,trans-dihydronepetalactone, and/or cis,cis-dihydronepetalactone, or a mixture of any two or more thereof is provided.

In a first process, dihydronepetalactone is produced by the hydrogenation of nepetalactone in the presence of a catalyst. In some embodiments, the catalyst contains nickel and may be selected from the group consisting of nickel, alloys thereof and compounds thereof. In some embodiments, the catalyst is a nickel-aluminum alloy (e.g., a Raney®-Nickel catalyst). A nickel-aluminum alloy may or may not alloyed with one or more additional metals. The catalyst may be undoped or doped with one or more dopants such as iron, molybdenum, chromium, zinc, tungsten, cobalt, manganese, and titanium. In some embodiments, the catalyst is elemental nickel. In some embodiments, the catalyst comprises elemental nickel and a nickel-containing alloy.

The catalyst may be supported or unsupported. If supported, a support such as silica, alumina, silica-alumina, zeolites, and activated carbon may be used. In some embodiments, an unsupported nickel-containing catalyst is used. In some embodiments, the catalyst is a sponge nickel catalyst. Specific examples include Raney®-Nickel catalysts and sponge nickel catalysts available from Strem Chemicals (CAS 7440-02-0).

The hydrogenation may further be effected in the presence of a promoter, including metal promoters and bases. Examples of metal promoters are tin, copper, gold, silver, molybdenum, iron, and combinations thereof. Examples of bases that may be used as promoters include sodium hydroxide, sodium methoxide, potassium hydroxide, potassium methoxide, as well as longer alkoxides. In some embodiments, hydrogenation is performed in the presence of an unsupported nickel-containing catalyst and a metal promotor. In some embodiments, hydrogenation is performed in the presence of an unsupported nickel-containing catalyst and a base.

The process may be performed with or without a solvent. Useful solvents include alcohols, amides, alkanes, esters, and ethers. Examples include ethyl acetate, butyl acetate, dimethylacetamide, ethanol, isopropyl alcohol, 1-butyl alcohol, 2-butyl alcohol, and dimethoxyethane.

Unexpectedly, it was found that the hydrogenation may be performed with water as a solvent. Thus, in some embodiments, the hydrogenation is performed in an aqueous medium. In some embodiments, a previous process step is performed in an aqueous medium. Performing hydrogenation in an aqueous medium can decrease the number of process steps required for industrial scale manufacturing.

The hydrogenation temperature may be from 25° C. to about 250° C., and in some embodiments, about 50° C. to about 150° C. Hydrogenation pressure may range from about 0.1 MPa to about 20 MPa.

According to various embodiments, at least 20%, at least 30%, at least 40%, at least 50%, at least 60%, at least 70%, at least 80%, at least 85%, at least 90%, at least 92%, at least 94%, at least 95%, at least 96%, at least 97%, at least 98%, at least 98.5%, at least 99%, at least 99.5%, at least 99.7%, at least 99.8%, at least 99.9%, at least 99.95%, at least 99.99%, or at least 100% of the nepetalactone is converted. Substantially all of the nepetalactone is considered converted if there is no detectable amount in the product.

Reaction time for an amount of nepetalactone to be converted may vary according to reaction temperature, catalyst, promoter, and reaction feed. Example reaction times are for at least 3 hours, 4 hours, 6 hours, 8 hours, 10 hours, 20 hours, or 24 hours. The dihydronepetalactone can be formed with a selectivity of at least 50%, at least 60%, at least 70%, at least 75%, at least 80%, at least 85%, at least 90%, at least 91%, at least 92%, at least 93%, at least 94%, or at least 95%.

### EXAMPLES

### Example 1: Hydrogenation of c,t-Nepetalactone in Ethanol and Room Temperature

Hydrogenation of c,t-nepetalactone (95% purity) with Raney®-Nickel 2800 (50% w/w) was carried out in ethanol at room temperature to produce c/t-dihydronepetalactone. Table 1, below, shows the results.

TABLE 1
Hydrogenation of nepetalactone with nickel
catalyst in ethanol at room temperature

| | | | Ethanol | | | | NPL | DHNPL |
|---|---|---|---|---|---|---|---|---|
| Scale | Yield | Solvent | (mL) | ° C. | Time | | (%) | isomers (%) |
| 2.0 g | 1.85 g | Ethanol | 8 mL | r.t | 20 | h | 0.75% | 4.3%/91.7% |
| 2.0 g | 1.84 g | Ethanol | 8 mL | r.t | 20 | h | 1.3% | 4.6%/90.5% |
| 2.0 g | — | Ethanol | 8 mL | r.t | 3 | h | 62.7% | 2.0%/31.3% |
| | | | | | 6 | h | 40.9% | 3.0%/50.7% |
| 2.0 g | 1.82 g | Ethanol | 24 mL | r.t | 20 | h | 3.3% | 4.1%/91.3% |
| | | | | | 48 | h | 0% | 4.8%/92.4% |

### Example 2: Hydrogenation of c,t-Nepetalactone with Nickel Catalyst in Ethyl Acetate and Ethanol at a Temperature of 60° C.

Hydrogenation of c,t-nepetalactone was performed in ethyl acetate and at temperature of 60° C. in 10% Raney®-Nickel 2800. After 20 hrs, remaining starting material was 2.2% (DHN isomer ratio 6.2%/88%), and after 26 hrs, it was 0.4%. When performed in ethanol at same conditions, remaining starting material was 5.5% after 20 hrs.

### Example 3: Hydrogenation of Nepetalactone with Nickel Catalyst and Different Solvents, Temperatures, and Pressures

Hydrogenation of nepetalactone with Raney®-Nickel was carried out with different solvents, temperatures, and pressures. 95% cis.trans-nepetalactone was hydrogenated to dihydronepetalactone under the conditions described below. Conversion and selectivity were measured from GC-FID. Selectivity is the sum of all dihydronepetalactone isomers. The results are shown in Table 2.

TABLE 2
Hydrogenation of nepetalactone with nickel catalyst and different solvents

| NPL | | | Cat | NPL | | Temp | H2 | | |
|---|---|---|---|---|---|---|---|---|---|
| (mg) | Cat/Time(h) | wt % | Wash | wt % | Solvent | C. | psig | Conv | Selec |
| 1098 | 2800/24 | 10 | Yes | 30 | EtOAc | 60 | 120 | 75 | 92 |
| | | | | | | | (OFF) | | |
| 618 | 2800/24 | 15 | Yes | 30 | EtOAc | 60 | 120 | 100 | 97 |
| | | | | | | | (OFF) | | |
| 514 | 2800/24 | 10 | Yes | 30 | EtOAc | 60 | 120 | 98.5 | 96 |
| 479 | 2800/24 | 10 | Yes | 30 | EtOAc | 80 | 120 | 100 | 96 |
| 444 | 2800/24 | 15 | Yes | 30 | EtOAc | 80 | 120 | 100 | 96 |
| 428 | 2800/24 | 15 | Yes | 30 | EtOAc | 60 | 120 | 99.6 | 92 |
| 402 | 2800/24 | 15 | Yes | 30 | EtOAc | 100 | 120 | 100 | 96 |
| 503 | 2800/24 | 10 | Yes | 30 | 2-BuOH | 60 | 120 | 99 | 95 |
| 475 | 2800/24 | 10 | Yes | 30 | 1-BuOH | 60 | 120 | 95 | 90 |
| 757 | 2800/24 | 10 | Yes | 30 | EtOAc | 80 | 120 | 99 | 94 |
| 583 | 2800/24 | 10 | Yes | 30 | EtOAc | 80 | 90 | 98 | 93 |
| 1466 | 2800/24 | 10 | Yes | 30 | EtOAc | 80 | 60 | 83 | 91 |
| 917 | 2800/24 | 10 | Yes | 30 | EtOAc | 80 | 120 | 95 | 92 |
| 537 | 2800/24 | 10 | Yes | 30 | 2-BuOH | 80 | 120 | 95 | 87 |
| 899 | 2800/24 | 10 | Yes | 30 | EtOAc | 80 | 120 | 100 | 93 |
| 984 | 2800/24 | 10 | Yes | 30 | 2-BuOH | 80 | 60 | 97 | 86 |
| 803 | 2800/24 | 10 | No | 30 | EtOAc | 80 | 150 | 100 | 95 |
| 573 | 2800/24 | 10 | Yes | 30 | EtOAc | 80 | 150 | 100 | 95 |
| 746 | 2800/24 | 10 | No | 30 | CH | 80 | 120 | 100 | 93 |
| 706 | 2800/24 | 10 | No | 30 | 2-BuOH | 80 | 150 | 100 | 92 |
| 702 | 2800/24 | 10 | Yes | 30 | DMAc | 80 | 120 | 92 | 92 |
| 769 | 2800/24 | 10 | Yes | 30 | EtOAc | 100 | 120 | 100 | 90 |
| 860 | 2800/24 | 10 | Yes | 30 | 2-BuOH | 100 | 120 | 100 | 85 |
| 820 | 2800/24 | 10 | Yes | 30 | IPA | 80 | 120 | 100 | 91 |
| 1069 | 2800/24 | 10 | Yes | 50 | IPA | 80 | 120 | 100 | 91 |
| 1010 | 2800/24 | 10 | Yes | 50 | EtOAc | 80 | 120 | 100 | 93 |
| 984 | 2800/24 | 10 | Yes | 50 | 2-BuOH | 80 | 120 | 100 | 89 |
| 1027 | 2800/24 | 10 | Yes | 50 | 1-BuOH | 80 | 120 | 100 | 86 |
| 852 | 2800/24 | 10 | Yes | 30 | 1-BuOH | 80 | 120 | 100 | 84 |
| 984 | 2800/24 | 10 | Yes | 30 | BuOAc | 80 | 120 | 100 | 94 |
| 320 | 2800/12 | 20 | No | 30 | EtOAc | 80 | 140 | 100 | 96 |
| 356 | 2800/12 | 20 | Yes | 30 | BuOAc | 80 | 140 | 100 | 94 |
| 426 | 2800/12 | 20 | No | 30 | EtOAc | 100 | 140 | 100 | 93 |
| 441 | 2800/12 | 20 | Yes | 30 | BuOAc | 100 | 140 | 100 | 91 |
| 351 | 2800/12 | 15 | No | 30 | EtOAc | 100 | 140 | 100 | 90 |
| 434 | 2800/12 | 15 | Yes | 30 | BuOAc | 100 | 140 | 100 | 89 |
| 428 | 2800/12 | 15 | No | 30 | EtOAc | 80 | 140 | 100 | 92 |
| 431 | 2800/12 | 15 | Yes | 30 | BuOAc | 80 | 140 | 100 | 92 |
| 668 | 2800/12 | 15 | Yes | 50 | H2O | 100 | 150 | 94 | 94 |
| 537 | 2800/12 | 15 | Yes | 50 | CH | 100 | 150 | 100 | 92 |
| 528 | 2800/12 | 15 | Yes | 50 | IPA | 100 | 150 | 100 | 90 |
| 561 | 2800/12 | 15 | Yes | 50 | DME | 100 | 150 | 100 | 89 |
| 969 | 2800/12 | 10 | Yes | 50 | H2O | 100 | 150 | 69 | 93 |
| 810 | 2800/12 | 10 | Yes | 50 | CH | 100 | 150 | 98.7 | 91 |
| 771 | 2800/12 | 10 | Yes | 50 | IPA | 100 | 150 | 100 | 89 |
| 693 | 2800/12 | 10 | Yes | 50 | DME | 100 | 150 | 93 | 88 |
| 665 | 2800/8 | 15 | Yes | 50 | BuOAc | 100 | 150 | 95 | 92 |
| 653 | 2800/8 | 15 | Yes | 50 | CH | 100 | 150 | 100 | 90 |
| 595 | 2800/8 | 15 | Yes | 50 | IPA | 100 | 150 | 100 | 88 |
| 690 | 2800/8 | 15 | Yes | 50 | BuOAc | 100 | 200 | 100 | 91 |
| 704 | 2800/8 | 15 | Yes | 50 | CH | 100 | 200 | 100 | 92 |
| 595 | 2800/8 | 15 | Yes | 50 | IPA | 100 | 200 | 100 | 88 |
| 1055 | 2800/8 | 10 | Yes | 50 | BuOAc | 100 | 200 | 100 | 91 |
| 910 | 2800/8 | 10 | Yes | 50 | IPA | 100 | 200 | 100 | 89 |
| 1079 | 2800/8 | 5 | No | 50 | BuOAc | 80 | 200 | 0 | ND |
| 690 | 2800/8 | 5 | No | 50 | BuOAc | 80 | 200 | 3 | ND |
| 1026 | 2800/8 | 5 | No | 50 | BuOAc | 80 | 225 | 60 | ND |
| 933 | 2800/8 | 5 | No | 50 | CH | 80 | 225 | 40 | ND |
| 719 | 2800/8 | 10 | No | 50 | CH | 80 | 200 | 90 | 94 |
| 944 | 2800/8 | 10 | No | 50 | IPA | 80 | 200 | 88 | 93 |
| 946 | 2800/8 | 10 | No | 50 | BuOAc | 70 | 200 | 80 | 93 |
| 784 | 2800/8 | 10 | No | 50 | CH | 70 | 200 | 76 | 92 |
| 1013 | 2800/6 | 10 | Yes | 50 | EtOAc | 100 | 200 | 99.5 | 91 |
| 791 | 2800/6 | 10 | Yes | 50 | EtOAc | 100 | 200 | 99 | 91 |
| 1440 | 2800/6 | 10 | Yes | 100 | Neat | 100 | 200 | 97.4 | 93 |
| 1146 | 2800/6 | 10 | Yes | 50 | H2O | 120 | 200 | 93 | 93 |
| 1197 | 2800/6 | 10 | Yes | 50 | EtOAc | 120 | 200 | 98.5 | 91 |
| 1190 | 2800/6 | 10 | Yes | 50 | EtOAc | 120 | 200 | 98.5 | 91 |
| 1282 | 2800/6 | 10 | Yes | 100 | Neat | 120 | 200 | 99.8 | 92 |
| 990 | 2800/6 | 12 | Yes | 50 | BuOAc | 100 | 200 | — | — |
| 940 | 2800/6 | 10 | Yes | 50 | BuOAc | 100 | 225 | — | — |
| 915 | 2800/6 | 10 | No | 50 | BuOAc | 100 | 225 | 99.2 | 91 |
| 926 | 2800/6 | 12 | No | 50 | BuOAc | 100 | 200 | 89.5 | 90 |
| 1006 | 2800/6 | 10 | Yes | 50 | IPA | 100 | 225 | — | — |
| 907 | 2800/6 | 12 | Yes | 50 | IPA | 100 | 200 | — | — |
| 1026 | 2800/6 | 10 | No | 50 | IPA | 100 | 225 | 100 | 89 |
| 893 | 2800/6 | 12 | No | 50 | IPA | 100 | 200 | 99.6 | 89 |
| 801 | 2800/6 | 10 | No | 50 | EtOAc | 100 | 225 | 90.5 | 90 |
| 1092 | 2800/6 | 10 | No | 50 | EtOAc | 100 | 225 | 94.2 | 90 |
| 1431 | 2800/6 | 10 | No | 75 | EtOAc | 100 | 225 | 90.4 | 91 |
| 1074 | 2800/6 | 10 | No | 75 | EtOAc | 100 | 225 | 97.6 | 92 |
| 1001 | 2800/6 | 10 | No | 50 | IPA | 100 | 225 | 97 | 90 |
| 1045 | 2800/6 | 10 | No | 50 | IPA | 100 | 225 | 99.1 | 90 |
| 1033 | 2800/6 | 10 | No | 75 | IPA | 100 | 225 | 97.2 | 91 |
| 1179 | 2800/6 | 10 | No | 75 | IPA | 100 | 225 | 96.7 | 90 |

As can be seen from the results in Table 1, hydrogenation of nepetalactone can be accomplished with Raney®-Nickel catalyst. Notably, the reaction can proceed in water with selectivity over 90%. Conversion in water can also be over 90%. The reaction also proceeds without the presence of a solvent with good reactivity and selectivity.

Without limiting the scope of the present disclosure, the following list represent exemplary embodiments:

Item 1. A process for the production of a dihydronepetalactone of formula (II) comprising hydrogenating a nepetalactone of formula (I) according to the following scheme:

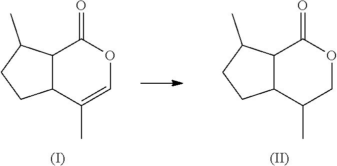

in the presence of a catalytic metal that includes nickel, wherein at least 20%, at least 30%, at least 40%, at least 50%, at least 60%, at least 70%, at least 80%, at least 85%, at least 90%, at least 92%, at least 94%, at least 95%, at least 96%, at least 97%, at least 98%, at least 98.5%, at least 99%, at least 99.5%, at least 99.7%, at least 99.8%, at least 99.9%, at least 99.95%, or at least 99.99% of the nepetalactone is converted.

Item 2. The process as recited in item 1, wherein the catalytic metal is selected from an unsupported catalytic metal, a nickel alloy, elemental nickel, or any combination thereof.

Item 3. The process as recited in item 1, wherein the catalytic metal consists essentially of a nickel-aluminum alloy.

Item 4. The process as recited in any of items 1-3, wherein the dihydronepetalactone is formed with a selectivity of at least 50%, at least 60%, at least 70%, at least 75%, at least 80%, at least 85%, at least 90%, at least 91%, at least 92%, at least 93%, at least 94%, or at least 95%.

Item 5. The process as recited in in any of items 1-4, wherein the hydrogenating is performed in the presence of a solvent.

Item 6. The process as recited in item 5, wherein the solvent is an alcohol, an amide, an alkane, an ester, or an ether.

Item 7. The process as recited in item 6, wherein the solvent is one of ethyl acetate, butyl acetate, dimethylacetamide, ethanol, isopropyl alcohol, 1-butyl alcohol, 2-butyl alcohol, and dimethoxyethane.

Item 8. The process as recited in item 5, wherein the solvent is water.

Item 9. The process as recited any of items 1-4, wherein the hydrogenating is performed without the presence of a solvent.

Item 10. The process as recited in any of items 1-9 wherein the process is effected in the presence of a metal promoter.

Item 11. The process as recited in item 10, wherein the metal promoter is selected from the group consisting of tin, copper, gold, silver, molybdenum, iron, and combinations thereof.

Item 12. The process as recited in any of items 1-11, wherein the hydrogenating is performed at a temperature of about 25° C. to about 250° C. and a pressure of about 0.1 MPa to about 20 MPa.

Item 13. The process as recited in any of items 1-12, wherein the hydrogenating is performed at temperature of about 50° C. to about 150° C.

Item 14. The process as recited in any of items 1-13, wherein the nepetalactone of Formula (I) is at least 85% cis/trans nepetalactone.

Item 15. A process for the production of a dihydronepetalactone of formula (II) comprising hydrogenating in an aqueous medium a nepetalactone of formula (I) according to the following scheme:

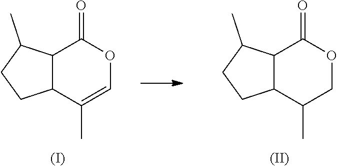

in the presence of a catalytic metal.

Item 16: The process as recited in item 15, wherein at least 20%, at least 30%, at least 40%, at least 50%, at least 60%, at least 70%, at least 80%, at least 85%, at least 90%, at least 92%, at least 94%, at least 95%, at least 96%, at least 97%, at least 98%, at least 98.5%, at least 99%, at least 99.5%, at least 99.7%, at least 99.8%, at least 99.9%, at least 99.95%, at least 99.99%, or at least 100% of the nepetalactone is converted.

Item 17. The process as recited in item 15 or 16, wherein the catalytic metal comprises nickel.

Item 18. The process as recited in item 17, wherein the catalytic metal consists essentially of a nickel-aluminum alloy.

Item 19. The process as recited in any of items 15-18, wherein the process is effected in the presence of a metal promoter.

Item 20. The process as recited in item 19, wherein the metal promoter is selected from the group consisting of tin, copper, gold, silver, molybdenum, iron, and combinations thereof.

Item 21. The process as recited in any of items 15-20, wherein the hydrogenating is performed at a temperature of about 25° C. to about 250° C. and a pressure of about 0.1 MPa to about 20 MPa.

Item 22. The process as recited in any of items 15-21, wherein the hydrogenating is performed at temperature of about 50° C. to about 150° C.

Item 23. The process as recited in any of items 15-23, wherein the nepetalactone of Formula (I) is at least 85% cis/trans nepetalactone.

1. A process for the production of a dihydronepetalactone of formula (II) comprising hydrogenating a nepetalactone of formula (I) according to the following scheme: 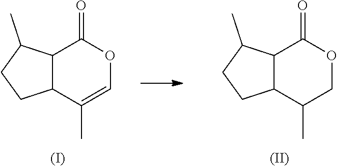
in the presence of a catalytic metal that includes nickel, wherein at least 20%, at least 30%, at least 40%, at least 50%, at least 60%, at least 70%, at least 80%, at least 85%, at least 90%, at least 92%, at least 94%, at least 95%, at least 96%, at least 97%, at least 98%, at least 98.5%, at least 99%, at least 99.5%, at least 99.7%, at least 99.8%, at least 99.9%, at least 99.95%, or at least 99.99% of the nepetalactone is converted.

2. The process as recited in claim 1, wherein the catalytic metal is selected from an unsupported catalytic metal, a nickel alloy, elemental nickel, or any combination thereof.

3. The process as recited in claim 1, wherein the catalytic metal consists essentially of a nickel-aluminum alloy.

4. The process as recited in claim 1, wherein the dihydronepetalactone is formed with a selectivity of at least 50%, at least 60%, at least 70%, at least 75%, at least 80%, at least 85%, at least 90%, at least 91%, at least 92%, at least 93%, at least 94%, or at least 95%.

5. The process as recited in claim 1, wherein the hydrogenating is performed in the presence of a solvent.

6. The process as recited in claim 5, wherein the solvent is an alcohol, an amide, an alkane, an ester, or an ether.

7. The process as recited in claim 6, wherein the solvent is one of ethyl acetate, butyl acetate, dimethylacetamide, ethanol, isopropyl alcohol, 1-butyl alcohol, 2-butyl alcohol, and dimethoxyethane.

8. The process as recited in claim 5, wherein the solvent is water.

9. The process as recited in claim 1, wherein the hydrogenating is performed without the presence of a solvent.

10. The process as recited in claim 1, wherein the process is effected in the presence of a metal promoter.

11. The process as recited in claim 10, wherein the metal promoter is selected from the group consisting of tin, copper, gold, silver, molybdenum, iron, and combinations thereof.

12. The process as recited in claim 1, wherein the hydrogenating is performed at a temperature of about 25° C. to about 250° C. and a pressure of about 0.1 MPa to about 20 MPa.

13. The process as recited in claim 1, wherein the hydrogenating is performed at temperature of about 50° C. to about 150° C.

14. The process as recited in claim 1, wherein the nepetalactone of Formula (I) is at least 85% cis/trans nepetalactone.

15. A process for the production of a dihydronepetalactone of formula (II) comprising hydrogenating in an aqueous medium a nepetalactone of formula (I) according to the following scheme: 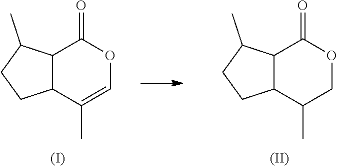
in the presence of a catalytic metal.

16. The process as recited in claim 15, wherein at least 20%, at least 30%, at least 40%, at least 50%, at least 60%, at least 70%, at least 80%, at least 85%, at least 90%, at least 92%, at least 94%, at least 95%, at least 96%, at least 97%, at least 98%, at least 98.5%, at least 99%, at least 99.5%, at least 99.7%, at least 99.8%, at least 99.9%, at least 99.95%, at least 99.99%, or at least 100% of the nepetalactone is converted.

17. The process as recited in claim 15, wherein the catalytic metal comprises nickel.

18. The process as recited in claim 17, wherein the catalytic metal consists essentially of a nickel-aluminum alloy.

19. The process as recited in claim 15, wherein the process is effected in the presence of a metal promoter.

20. The process as recited in claim 19, wherein the metal promoter is selected from the group consisting of tin, copper, gold, silver, molybdenum, iron, and combinations thereof.

21. The process as recited in claim 15, wherein the hydrogenating is performed at a temperature of about 25° C. to about 250° C. and a pressure of about 0.1 MPa to about 20 MPa.

22. The process as recited in claim 15, wherein the hydrogenating is performed at temperature of about 50° C. to about 150° C.

23. The process as recited in claim 15, wherein the nepetalactone of Formula (I) is at least 85% cis/trans nepetalactone.
